(19) United States
(12) Patent Application Publication
(10) Pub. No.: US 2005/0080428 A1
(43) Pub. Date: Apr. 14, 2005

(54) Extracapsular surgical procedure for repair of anterior cruciate ligament rupture and surgical referencing instrument therefor

(76) Inventor: Ralph Richard White, Portland, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 10/934,269

(22) Filed: Sep. 3, 2004

Related U.S. Application Data
(60) Provisional application No. 60/499,859, filed on Sep. 3, 2003.

(51) Int. Cl. A61B 17/58

(52) U.S. Cl. 606/102

(57) ABSTRACT

An instrument for measuring the isometric points in the joint of a mammal is provided.

This application claims the benefit of U.S. provisional patent application Ser. No. 60/499,859, filed Sep. 3, 2003.

### BACKGROUND OF THE INVENTION

Current surgical techniques for anterior ligament replacement and/or stabilization are grouped into extracapsular procedures and intracapsular procedures. The extracapsular procedures use tissues or suture implants placed outside of the joint capsule in order to stabilize the joint. Heavy sutures (modified Flo imbrication), and the repositioning of the lateral collateral ligament (fibular head transposition), are the current accepted techniques in veterinary surgery.

### BRIEF SUMMARY OF THE INVENTION

Disclosed is a surgical procedure for extra capsular anterior ligament replacement or stabilization and a specialized surgical instrument therefor.

Intracapsular procedures require a graft from an adjacent tissue such as the straight patellar tendon or the fascia lata which is detached from its origin and inserted or repositioned through tunnels bored in the distal femur and/or proximal tibia. This surgery is done inside of the joint capsule, with both ends of the transplant being fixed to the walls of the tunnels and/or adjacent bone using techniques of the particular surgeon's choosing.

Instruments for determining the isometric points of an intracapsular graft attachment are well known in the art. U.S. Pat. Nos. 5,037,426 and 5,743,909 teach instruments that represent the current art for the intracapsular determination of isometric points of attachment of these grafts. However, the current extracapsnlar surgical procedures have not addressed the optimal placement of transplant tissue or suture at the isometric points. Accordingly, it is to be understood that the surgical procedure of this invention provides for the optimal placement of these tissues in an extracapsular procedure utilizing the surgical referencing instrument of this invention to identify the isometric points of attachment.

### BRIEF DESCRIPTION OF THE SEVERAL VIEWS OF THE DRAWINGS

FIG. 1 is a perspective view of a preferred embodiment of the surgical instrument of this invention shown in approximately double scale.

FIG. 2 is a side elevation of the instrument of FIG. 1 shown in approximately full scale.

FIG. 3 is a side elevation of the instrument of FIG. 1 with the referencing block members removed.

FIG. 4 is a side elevation of the adjustable block member separated from the threaded rod of the instrument and showing the threaded, central mounting bore extending therethrough.

FIG. 4A is a top plan view of the block member of FIG. 4 as viewed from the top in FIG. 4.

FIG. 5 is a side elevation of the stationary block member separated from the non-threaded rod portion of the surgical instrument of this invention and showing the centrally disposed, non-threaded mounting bore extending therethrough and a pair of auxiliary-use bores provided therethrough.

FIG. 5A is a top plan view of the stationary block member of FIG. 5 as viewed from the top in FIG. 5.

FIG. 6 is a perspective view of an alternate embodiment of the surgical instrument of this invention.

FIG. 7 is an additional perspective view of an alternate embodiment of the surgical instrument of this invention..

FIGS. 8 and 9 are fragmentary schematic side elevations of a dog knee joint in full extension and full flexion respectively, and showing the reference and isometric points in each condition of the joint.

FIGS. 10 and 11 are perspective views of an alternate embodiment of this invention.

FIG. 12 is a side elevation of the instrument of FIGS. 10 and 11.

FIG. 13 is a top view of the instrument of FIGS. 10 and 11.

FIG. 14 is a front view of the instrument of FIGS. 10 and 11.

### DETAILED DESCRIPTION OF THE INVENTION

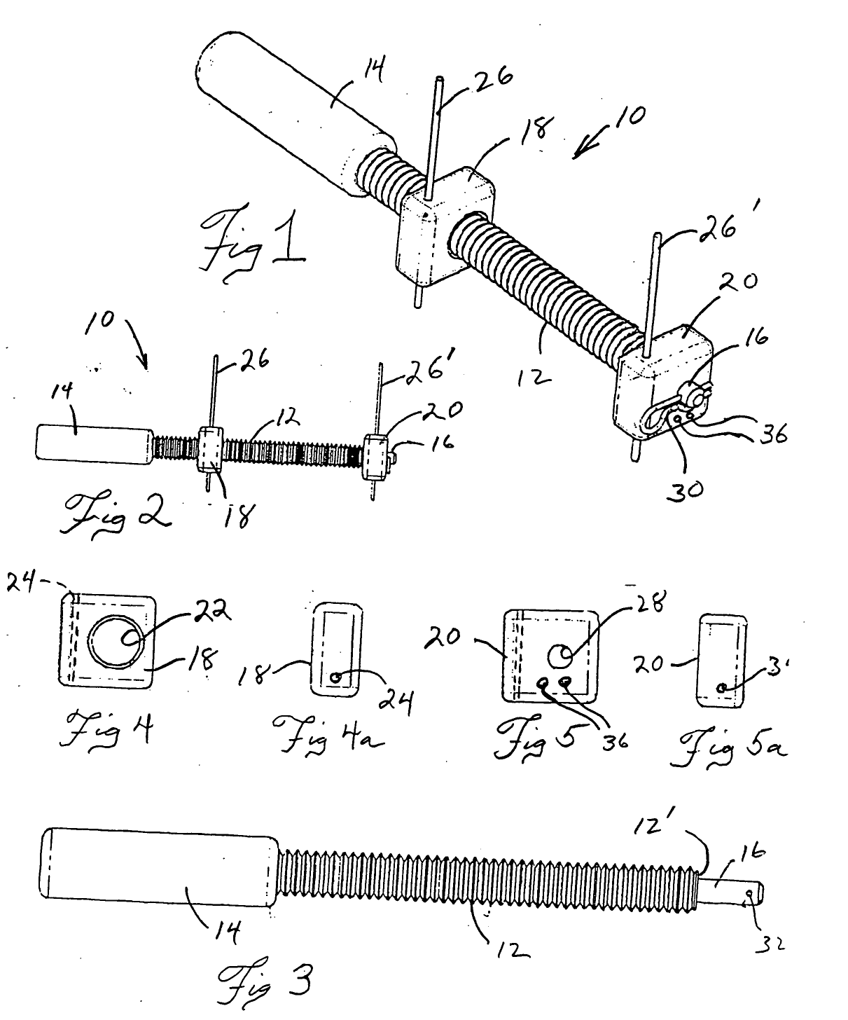

First, with regard to the surgical instrument 10 of this invention, there is shown in FIGS. 1-5A a preferred embodiment which utilizes an extremely simple yet extremely efficient tool construction. In this, a longitudinally-elongated rod member is provided with a longitudinally extending threaded portion 12 having a predetermined overall length and predetermined thread pattern. As illustrated, the rod terminates at one of its ends in a hand-grasp handle portion 14 configured to facilitate the holding and rotating of the surgical instrument in use as will become clear later. The opposite terminal end of the rod includes an axially-projecting, non-threaded shaft portion 16 shown in this particular embodiment as having a reduced diameter relative to the threaded portion 12 of the rod member. As will be understood by those skilled in the art, the entire rod assembly 12, 14, 16 described thus far may if desired be formed as a unitary, integral member for simplicity of manufacture, of any suitable material, such as metal, capable of being repeatedly and properly sterilized as required for use in surgical procedures.

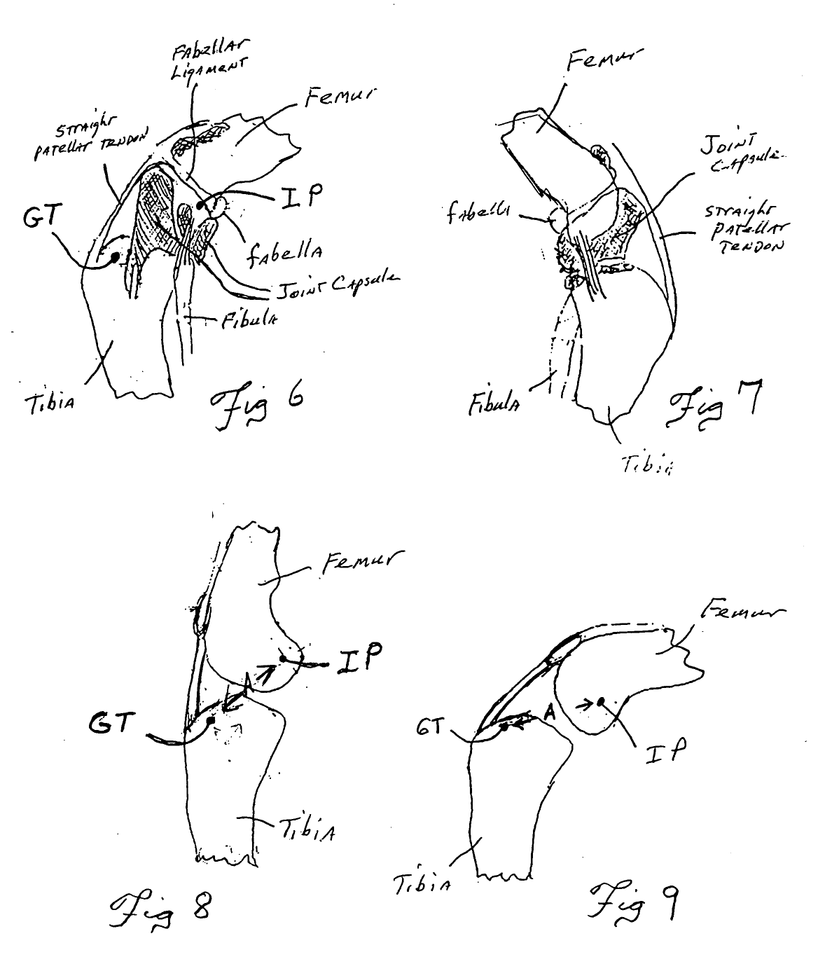

As is evident in viewing FIGS. 1 and 2 of the drawings, the rod member 5 mounts a pair of wire-supporting referencing members, illustrated herein as block members 18, 20 configured for operative support on the threaded portion 12 and non-threaded shaft portion 16, respectively, of the rod member. In this regard, and as can be best seen in FIG. 4, block member 18 is provided as an adjustable block member having a threaded mounting bore 22 therethrough configured to 10 cooperatively correspond with the threaded rod portion 12 of the instrument, whereby the adjustable block member 18 may be threaded onto and along the threaded portion 12 of the rod as is readily apparent. This adjustable block member 18 is also provided with a wire-receiving bore 24 therethrough extending along an axis that is substantially perpendicular to the axis of the threaded bore 22 and, in turn, the axis of the rod when the block is threadedly mounted on the threaded portion 12, as is readily apparent in viewing FIGS. 1 and 2 of the drawings. The diameter of this bore 24 is selected to provide for sliding reception of selected pins and wires such as a K-wire 26, a Steinmann pin, and others known to those skilled in the surgical art.

Block 20, here referred to as stationary block 20 may, as illustrated, be configured generally similar to the aforementioned adjustable block 18 with the exception that the mounting bore 28 extending through the block body is configured for free, rotatable reception on the non-threaded, reduced diameter shaft portion 16 of the rod member. As is readily apparent in viewing the drawings, this block member 20 is rotatably captured on the reduced diameter shaft portion 16 of the rod member between the abutting enlarged end 12′ of the threaded portion and a cotter pin 30 releasably engaged through a bore 32 provided through the shaft portion adjacent its outer terminal end. Also, the block member 20 includes a wire receiving bore 34 therethrough similar to the bore 24 described in connection with the adjustable block member 18. As seen in FIGS. 1 and 5 of the drawings, the block 20 may also be provided with at least one, and in the embodiment illustrated, a pair of bores 36 extending through the block member on an axis substantially parallel to the axis of the bore 28 through the block member. These bores 36 may be provided for a purpose to be described.

From the foregoing it will be apparent that the instrument described thus far provides a tool whereby one referencing element, block member 20, is, rotatably secured in a stationary position adjacent the end of the elongated rod member, and a second referencing member, block member 18, is supported for adjustment toward and away from the first referencing member. In the particular embodiment illustrated, it will be apparent that, with block member 18 held against rotation, rotation of the rod, as by handle member 14, in different directions effectively threads the block member 18 correspondingly in opposite directions towards and away from the block member 20.

Having thus described the basic structure of a preferred embodiment of the surgical instrument of the present invention, the surgical procedure and the operation of the surgical instrument in the surgical procedure will now be described in connection with a procedure on the knee joint of a dog.

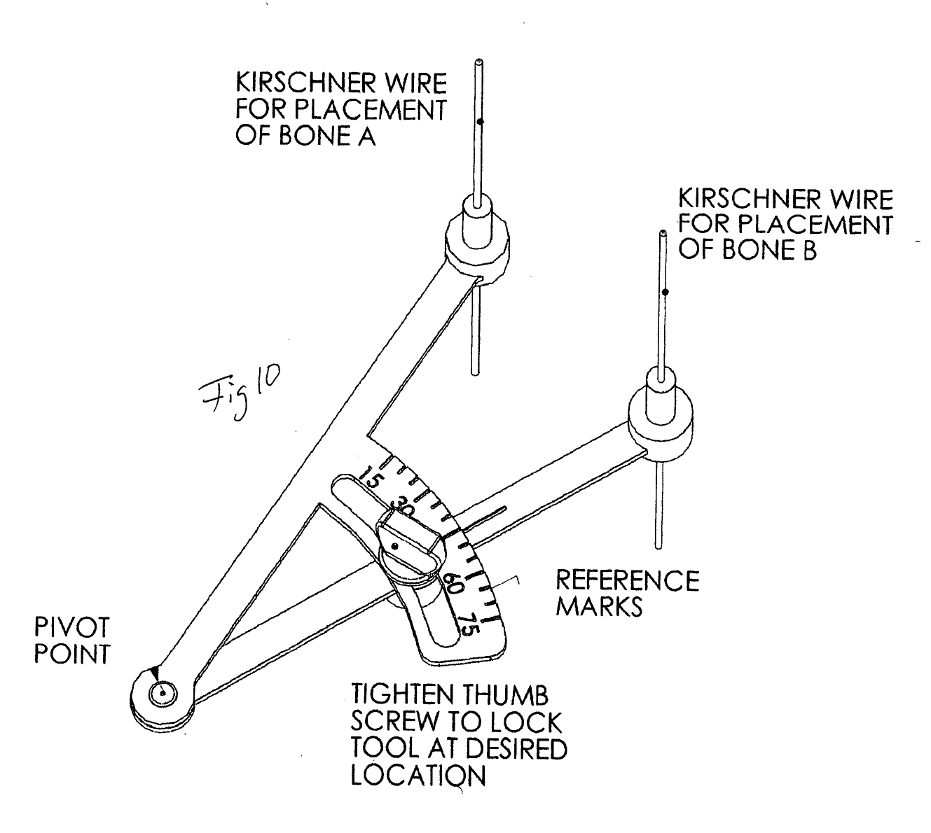

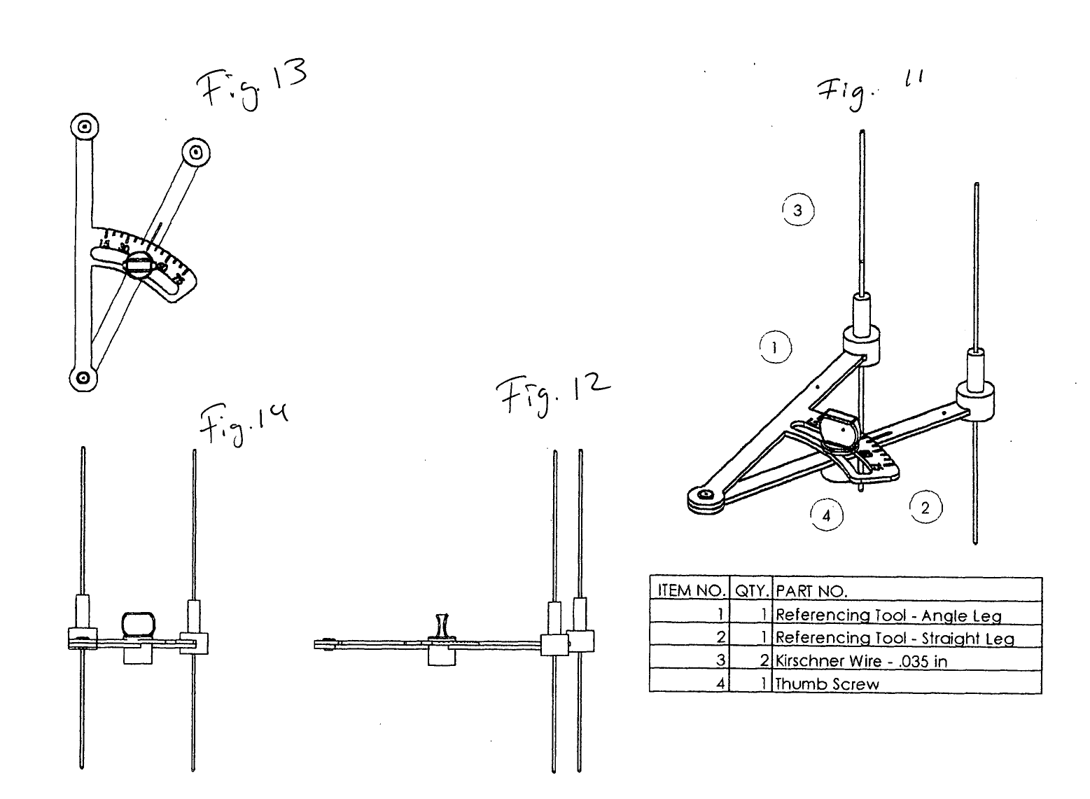

First, full scale or known scale latero-medial radiographs are taken of both knees in full flexed position and extended position. Using the latero-medial extended view, Gerdy's tubercle (GT) is identified and used as the tibial isometric reference point. This point is referenced on both radiographs with a marker. Using a standard compass, the center leg point is placed at GT. A first point is located antero-ventral to the lateral femoral fabella on the lateral femoral condyle, and the compass is adjusted to that distance. This process is repeated on the latero-medial flexed view. By repeating this process back and forth between radiographs of the joint in extended and flexed positions, making minor adjustments in the compass reference points, the optimal isometric point IP may be identified on the femur at the point at which the distance A between the finally identified point on the femur and the reference point GT is substantially identical on the radiographs of the joint with the knee in fully extended and flexed conditions as is indicated in FIGS. 8 and 9 of the drawings. The distance between the points of the compass determines the isometric distance A.

The isometric referencing instrument is adjusted so that the center of the bores 24 and 34 on the block members 18 and 20 equal the isometric distance A previously determined by the aforementioned compass method. The isometric referencing instrument is then sterilized for surgery.

An anterorlateral surgical approach along the lateral margin of the straight patellar tendon and vastus lateralis muscle is made to expose the tibial crest, lateral margin of the patellar tendon, patella and distal vastus lateralis. The lateral border of the straight patellar tendon, patella and lateral body of the vastus lateralis muscle are identified and carefully dissected free from the underlying joint capsule, lateral patella ligament, and adjacent biceps femoris muscle. This identifies the anterior border of the fascia lata and exposes the tendonous insertion of the fascia lata on the proximal tibia at Gerdy's tubercle (GT). A lateral or medeal arthrotomy is made to inspect the joint contents. Appropriate procedures are completed and the joint is flushed and closed.

A 1.5 mm K-wire 26 is inserted into the bore 24 through block 18 and directed perpendicularly to the long axis of the tibia into the tibial tuberosity at Gerdy's tubercle GT at the tendenous insertion of the fascia lata, being careful to avoid the joint space. The K-wire is then seated in conventional manner deep into the tibia once it has been determined that it is being placed accurately at Gerdy's tubercle GT. This temporary anchoring of the K-wire 26 at GT defines, by the anchored wire, the distal isometric reference point on the tibia located between the insertion of the straight patellar tendon and the muscular groove. The instrument is then positioned on the seated wire by positioning the bore 24 of the block member 18 over the exposed end of the wire and lowering the instrument slidingly down onto the wire. The knee is placed in extension and a blunted 2.5 mm (3/32″) Steinmann Pin 26′ is inserted through the bore 34 in the block member 20.

Having already predetermined and preset the isometric distance A between the bores 24, 34 of the block members 18, 20 during the aforementioned procedure with the radiographs, the femoral isometric point IP is located on the lateral femoral condyle using a trial and error positioning of the knee joint between flexion and extension. When the precise isometric point is determined, the tension corresponding to the isometric distance A reference will be zero at maximum flexion and extension. Therefore, the identified IP point on the femur is the common point at which the Steinmann Pin contacts the femur when the leg is in each of its extended and flexed conditions with the surgical instrument supported on the seated K-wire anchored at Gerdy's tubercle GT.

The purpose, of course, of using a blunted Steinmann pin while ascertaining the IP point is to assure against tearing or damage to the soft tissues in the area particularly as the leg is moved between extension and flexion. With the IP point thus located, the blunted Steinmann pin is removed from the bore 34 and replaced with a shortened 2.5 mm (3/32″) Steinmann trocar tipped pin, which is driven into the lateral femoral condyle point at the isometric point IP to a depth of approximately 10 mm. The pin is left in place temporarily to mark the femoral isometric point. The referencing instrument is then removed from the Steinmann trocar tip pin and K-wire 26. The distal K-wire 26 may also be removed from its seated mount on the tibia at Gerdy's tubercle GT.

A second incision is begun at a point 5-15 mm caudal to the tendonous insertion of the fascia lata at Gerdy's tubercle GT. This incision is extended with blunt dissection along the fascial lines of the fascia lata and the caudal aspect of the vastus lateralis muscle to the isometric point IP on the femur previously referenced by the shortened Steinmann Pin. The incision is extended proximally for about 15 mm. The tissue transplant is freed from deeper structures by blunt dissection. The lateral patellar ligament and insertion of the long digital extensor tendon are preserved and the transplant is freed distally to the level of Gerdy's tubercle.

A periosteal H-plasty is performed at the Steinmann Pin located at IP. The cortical bone that underlies the H-plasty is roughened with an elevator to encourage new bone growth into the graft. A 2.7 mm bone anchor is threaded with two pieces of non-absorbable monofilament nylon sutureleaving four strands of suture. The Steinmann Pin is then removed and the 3.5 mm bone anchor is driven into the femoral condyle at the isometric point IP. One piece (2 strands) will be used to apply traction to the transplant and transfix the transplant to the bone anchor.

Once the bone anchor is seated and the H-plasty is performed with the flaps retracted with appropriate suture material, the tool of this invention may be used in a second capacity to apply traction to the transplant thusly: A stab incision is performed in a linear fashion over and proximal to the anchor in the fascia lata transplant. This allows the anchor and the strands of suture material to protrude through the incision and transplant. A strand of suture is passed through the holes (36) in the proximal block (20) and transfixed to the graft at a point approximately 15 mm distal to the bone anchor in place. The screw mechanism is adjusted such that block (20) moves away from the bone anchor, thus placing the transplant in tension. Once all joint instability is eliminated with this adjustment, the suture strands in place through the bone anchor are used to transfix the transplant and maintain the tension on the transplant. The “tension” suture is severed, releasing the block from the graft and the graft is tested for tension through range of motion. The joint is tested for stability. Once the joint is stable through range of motion, the retaining sutures on the H-plasty are used to transfix the fascial lata transplant in place.

This traction suture places the graft in moderate tension. At this point in the surgery, joint stability should be restored in full flexion and extension. The other piece of suture is likewise used to transfix the transplant immediately distal the bone anchor. Finally the flaps of the periosteal H-plasty are sutured in a transfixing manner to the graft such that the periosteal flaps wrap around the transplant and ensure that the transplant is secured to the periosteum and is in direct contact with cortical bone of the lateral femoral condyle.

A Flo-type suture may then be placed in the traditional manner and aligned with both strands parallel to the fascia lata transplant. The distal fixation bore holes on the distal straight patellar tendon and tibial crest are determined such that the suture remains parallel to the transplant throughout the entire range of motion 5 of the joint. This minimizes the chance for the Flo-type suture to interfere with, or injure the transplant.

The suture is tightened until joint instability is eliminated throughout the entire range of motion of the joint. The tensioned suture is tested for isometric correctness, ensuring that the tensioned suture has not affected the isometric distance previously determined. This suture acts as a bridge that protects the fascia lata transplant during the re-vascularization and strengthening of that suture. The suture will ultimately fail, leaving the isometric transplant to function as the extra capsular anterior cruciate ligament. The tissues are then finally closed in typical manner.

It will be apparent to those skilled in the art that various changes can be made in the procedure and techniques described hereinbefore with respect to the surgical procedure as may be desired or needed in different cases. Also, it will be apparent to those skilled in the art that various changes, other than those discussed hereinbefore may be made in the size, shape, type, number and arrangement of the parts and structures of the surgical instrument described hereinbefore without departing from the spirit of the invention.

1. A device for measuring the isometric points in the joints of non-human mammals comprising:
(a) two arms having terminal ends extending from a pivot point,
(b) each of said terminal ends having a receptacle for a marking apparatus,
(c) said arms being slideably interconnected by a slotted member extending between said arms at a point between said pivot point and said terminal ends, said slotted member being selectively lockable at a pre-determinable point.
